(19) United States
(12) Patent Application Publication
(10) Pub. No.: US 2024/0194317 A1
(43) Pub. Date: Jun. 13, 2024

(54) METHOD, APPARATUS RELATED TO MEDICATION LIST MANAGEMENT, AND COMPUTER-READABLE RECORDING MEDIUM

(71) Applicant: AESOP Technology Inc., San Francisco, CA (US)

(72) Inventors: An Jim Long, Taipei City (TW); Yu-Chuan Li, New Taipei City (TW); Yi Hsiu Lin, Taipei City (TW)

(73) Assignee: AESOP Technology Inc., San Francisco, CA (US)

(21) Appl. No.: 18/286,750

(22) PCT Filed: Nov. 25, 2021
(86) PCT No.: PCT/US2021/060881

§ 371 Date: Oct. 12, 2023

Related U.S. Application Data
(60) Provisional application No. 63/196,186, filed on Jun. 2, 2021.

(30) Foreign Application Priority Data
Jul. 5, 2021 (TW) 110124537

(51) Int. Cl. G16H 20/10; G16H 10/60

(52) U.S. Cl. CPC G16H 20/10; G16H 10/60

(57) ABSTRACT

A method and an apparatus related to medication list management are provided. The medication association between one or more diagnoses and one or more corresponding medications recorded in a medical record is determined through an evaluating model. The evaluating model is trained through a machine learning algorithm. Multiple medication categories are integrated into the medical record based on the medication association. The medication includes one or both of a first medication and a second medication. The medication categories include an explained category related to the first medication with higher medication association and an unexplained category related to the second medication with lower medication association. A medication list interface presenting the medical record with multiple medication categories is provided. Accordingly, the medication history would be structured, grouped, and visual-encoded, so as to provide an intuitive medication list.

### CROSS-REFERENCE TO RELATED APPLICATION

This application claims the priority benefits of US application Ser. No. 63/196,186, filed on Jun. 2, 2021 and Taiwan application serial no. 110124537, filed on Jul. 5, 2021. The entirety of the above-mentioned patent applications are hereby incorporated by reference herein and made a part of this specification.

### BACKGROUND OF THE DISCLOSURE

### 1. Field of the Disclosure

The present disclosure generally relates to medication management in particular, to a method, an apparatus related to medication list management, and a computer-readable recording medium.

### 2. Description of Related Art

A traditional electronic medical record provides merely a timing sequence for medication list management. For example, table (1) is a medical record. The objectives (such as medical imaging tests and outpatient drugs) and facilities (such as hospital A and hospital E) are merely associated with dates and become group units of the medical record.

TABLE (1)

| Objective | facility | Date |
|---|---|---|
| Medical imaging reports | Hospital A | 2016 May 29 15:00 |
| Outpatient drug record | Hospital E | 2016 May 24 10:25 |
| Laboratory test reports | Hospital A | 2016 May 5 22:15 |
| . . . | . . . | . . . |
| Discharge summaries | Hospital D | 2016 Jan. 28 20:32 |

It should be noticed that it is hard to figure out a long-term medication trend from the traditional electronic medical record by the manner of using the timing sequence as a group unit. It is impossible to perform cross-checking on medication from the traditional electronic medical record having multiple visit records within the same duration. Furthermore, if a doctor needs to check a medication list, he/she has to select each medical record at different time points one by one. Therefore, it would bring a burden for clinical persons with heavy work loading.

### SUMMARY OF THE DISCLOSURE

Accordingly, the present disclosure is directed to a method, an apparatus related to medication list management, and a computer-readable recording medium.

In one of the exemplary embodiments, a method related to medication list management includes, but is not limited thereto, the following steps. The medication association between one or more diagnoses and one or more corresponding medications recorded in a medical record is determined through an evaluating model. The evaluating model is trained through a machine learning algorithm. Multiple medication categories are integrated into the medical record based on the medication association. The medication includes one or both of a first medication and a second medication. The medication categories include an explained category related to the first medication with higher medication association and an unexplained category related to the second medication with lower medication association. A medication list interface presenting the medical record with multiple medication categories is provided.

In one of the exemplary embodiments, an apparatus, includes, but is not limited thereto, a memory, a display, and a processor. The memory is used for storing program code. The processor is coupled to the memory and the display. The processor is coupled to the display and the memory. The processor is configured for loading and executing the program code to perform the following steps. The medication association between one or more diagnoses and one or more corresponding medications recorded in a medical record is determined through an evaluating model. The evaluating model is trained through a machine learning algorithm. Multiple medication categories are integrated into the medical record based on the medication association. The medication includes one or both of a first medication and a second medication. The medication categories include an explained category related to the first medication with higher medication association and an unexplained category related to the second medication with lower medication association. A medication list interface presenting the medical record with multiple medication categories is provided through the display.

In one of the exemplary embodiments, a non-transitory computer-readable recording medium, records a program code. The program code is loaded onto a processor to perform the following steps. The medication association between one or more diagnoses and one or more corresponding medications recorded in a medical record is determined through an evaluating model. The evaluating model is trained through a machine learning algorithm. Multiple medication categories are integrated into the medical record based on the medication association. The medication includes one or both of a first medication and a second medication. The medication categories include an explained category related to the first medication with higher medication association and an unexplained category related to the second medication with lower medication association. A medication list interface presenting the medical record with multiple medication categories is provided through the display.

It should be understood, however, that this Summary may not contain all of the aspects and embodiments of the present disclosure, is not meant to be limiting or restrictive in any manner, and that the invention as disclosed herein is and will be understood by those of ordinary skill in the art to encompass obvious improvements and modifications thereto.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a block diagram illustrating an apparatus according to one of the exemplary embodiments of the disclosure.

FIG. 2 is a flowchart illustrating a method related to medication list management according to one of the exemplary embodiments of the disclosure.

FIG. 3 is a schematic diagram illustrating medication categories related to association according to one of the exemplary embodiments of the disclosure.

FIG. 4 is a schematic diagram illustrating medication categories according to one of the exemplary embodiments of the disclosure.

FIG. 5 is a schematic diagram illustrating guiding information related to unexplained medication according to one of the exemplary embodiments of the disclosure.

FIG. 6 is a schematic diagram illustrating guiding information related to unexplained diagnosis according to one of the exemplary embodiments of the disclosure.

FIG. 7 is a schematic diagram illustrating guiding information related to drug interaction according to one of the exemplary embodiments of the disclosure.

### DESCRIPTION OF THE EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

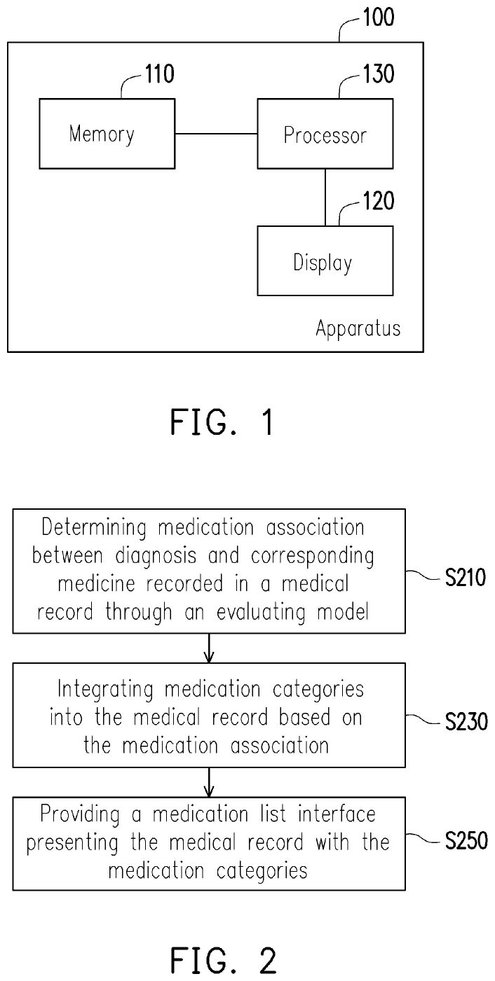

FIG. 1 is a block diagram illustrating an apparatus 100 according to one of the exemplary embodiments of the disclosure. Referring to FIG. 1, the apparatus 100 includes, but is not limited thereto, a memory 110, a display 120, and a processor 130. In one embodiment, the apparatuses 100 could be a computer, a server, a smartphone, a tablet computer, a wearable device, a personal assistant, or the likes. In some embodiments, the apparatus 100 is adapted for medical or clinical-related technologies.

The memory 110 may be any type of fixed or movable random-access memory (RAM), a read-only memory (ROM), a flash memory, a similar device, or a combination of the above devices. In one embodiment, the memory 110 is used to store program codes, device configurations, buffer data, or permanent data (such as medical record, medication association, or evaluating model), and these data would be introduced later.

The display 120 may be an LCD, a LED display, or an OLED display. In one embodiment, the display 120 is used to present a graphical interface.

The processor 130 is coupled to the display 120 and the memory 110. The processor 130 is configured to load and execute the program code(s) stored in the memory 110, to perform a procedure of the exemplary embodiment of the disclosure.

In some embodiments, the processor 130 may be a central processing unit (CPU), a microprocessor, a microcontroller, a graphics processing unit (GPU), a digital signal processing (DSP) chip, a neural network accelerator, or a field-programmable gate array (FPGA). The functions of the processor 150 may also be implemented by an independent electronic device or an integrated circuit (IC), and operations of the processor 130 may also be implemented by software.

To better understand the operating process provided in one or more embodiments of the disclosure, several embodiments will be exemplified below to elaborate the apparatus 100. The elements, units, and modules in apparatus 100 are applied in the following embodiments to explain the method related to medication list management provided herein. Each step of the method can be adjusted according to actual implementation situations and should not be limited to what is described herein.

FIG. 2 is a flowchart illustrating a method related to medication list management according to one of the exemplary embodiments of the disclosure. Referring to FIG. 2, the processor 130 determines medication association between one or more diagnoses and one or more corresponding medications recorded in a medical record through an evaluating model (step S210). Specifically, the medical record (also called a health record or a medical chart) is an electronic medical record or a digital format of a medical record. An original medical record may have the categories such as diagnosis, medication, and date. Some medical records may have sorted diagnoses or medications based on the order of the dates.

On the other hand, the evaluating model is trained through a machine learning algorithm. The machine learning algorithm may be a supervised learning algorithm or an unsupervised learning algorithm. The machine learning algorithm may analyze training samples to obtain a pattern from the training samples, so as to predict unknown data through the pattern. The evaluating model is a machine learning model constructed after being trained, and inferences on the data to be evaluated are made based on the evaluating model.

In an embodiment, the evaluating model uses actual prescription medications and diagnoses from one or more medical records as the training samples. In addition, the medication association relates to the degree or coefficient of association between medications and diagnoses. For example, a higher medication association indicates a higher degree of association between a medication and a diagnosis, and may mean that the medication is included in most of the prescriptions for the diagnosis (but not limited to thereto). Alternatively, a lower medication association indicates a lower degree of association between a medication and a diagnosis, and may mean that the medication is not included in all the prescriptions for the diagnosis (but not limited to thereto).

It should be noted that the aforementioned “higher medication association” and “lower medication association” may be determined based on a threshold related to an actual number or amount (but not limited to thereto).

In an embodiment, the evaluating model is a probabilistic model. The probabilistic model is an unsupervised learning algorithm and is an important method of data mining.

In another embodiment, the evaluating model is a neural network model. For example, a deep neural network (DNN). This deep neural network architecture includes an input layer, a hidden layer, and an output layer. It is to be noted that the deep neural network is formed by a multi-layer neuron structure, and each layer of neurons is configured with an input (for example, an output of a previous layer of neurons) and an output. The neurons in any layer of the hidden layer, through the inner product of an input vector and a weight vector, output a scalar result through a nonlinear transfer function. In the learning stage of the evaluating model, the aforementioned weight vector is trained and determined. Alternatively, while in the inference stage of the evaluating model, the determined weight vector is used to obtain an evaluation result (that is, the output). In this embodiment, the evaluation result of the evaluating model is the medication association between input variables. The medication association may be a probability, Q coefficient, or other quantitative values. The input variables include, for example, medications, diagnoses, diseases, patient characteristics (for example, gender, age, race, socioeconomic status, or weight), and/or visit facilities.

In some embodiments, the medication association outputted from the evaluating model may be further optimized by maintaining the higher medication association and disconnecting the lower medication association.

Referring to FIG. 2, the processor 130 integrates multiple medication categories into the medical record based on the medication association (step S230). Specifically, the medication includes one or both of a first medication and a second medication. The first medication is the medication having higher medication association with its corresponding diagnosis. The second medication is the medication having lower medication association with its corresponding diagnosis. The medication categories include an explained category related to the first medication with higher medication association and an unexplained category related to the second medication with lower medication association. That is, the first medication would be classified into the explained category, and the second medication would be classified into the unexplained category. The explained category relates to medication that could be explained by diagnosis, inspection, check, or surgery, so that the medication is ordered without any doubt. On the other hand, the unexplained category relates to medication that could not be explained by diagnosis, inspection, check, or surgery, so that the medication is ordered with doubt and should be clarified or intervened.

In one embodiment, the unexplained category may be further divided into an unexplained medication sub-category and an unexplained diagnosis sub-category. The unexplained medication sub-category relates to medication that could not be explained. The unexplained diagnosis sub-category relates to a diagnosis that could not be explained. Furthermore, some unexplained medications whose treatment such as anti-inflammatory, analgesic, or external use could be classified into low-risk medication.

In one embodiment, the medication categories further include an unmedicated category. The processor 130 may determine a diagnosis that has no medication recorded in the medical record belongs to the unmedicated category. For example, in a diagnosis, hypokalemia may be considered with a potassium ion supplement, so as to prevent the heart from being impacted. Therefore, if this diagnosis is ordered without any corresponding treatment or medication, the diagnosis should be clarified or intervened.

In one embodiment, the medication categories further include a drug interaction category. It is assumed that there may be multiple medications recorded in the medical record. The processor 130 may determine the interaction between these medications recorded in the medical record. The medication with the interaction relating to duplicating medication or lowering efficacy belongs to the drug interaction category. For example, the interaction between hypolipidemic medication and steroids may lower the efficacy of the hypolipidemic medication. Therefore, hypolipidemic medication and steroids would be classified into the drug interaction category. The interaction of medications may be determined based on literature or databases.

In one embodiment, the medication categories further include a low-risk category. The processor 130 may determine a side effect of the medication recorded in the medical record. The medication having side effects with less harm belongs to the low-risk category. For example, if the medication is an injection or a treatment for specific symptoms, for example, fever reduction or inflammation reduction, an individual medication would not harm a patient. The side effect of medication may be determined based on literature or databases.

In some embodiments, more medication categories may be integrated into the medical record based on actual requirements. For example, a category related to dosage mistakes or recent medications may be added to the medical record.

Therefore, not only date, more category units would be added to the medical record.

Referring to FIG. 2, the processor 130 provides a medication list interface presenting the medical record with the medication categories through the display 120 (step S250). Specifically, to provide an intuitive manner, a graphical interface including multiple medication categories such as the explained category, unexplained category, or the unmedicated category could be presented on the display 120.

In one embodiment, the processor 130 may provide one or more blocks on the medication list interface. Each block corresponds to one medication category. Different blocks would be located at different areas on the medication list interface. That is, two medications belonging to different medication categories would be separated into different blocks on the medication list interface.

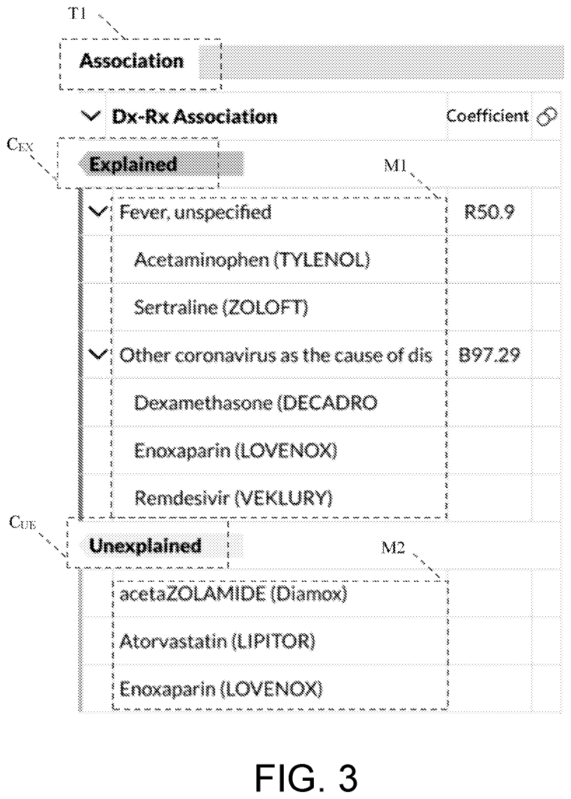

For example, FIG. 3 is a schematic diagram illustrating medication categories related to association according to one of the exemplary embodiments of the disclosure. Referring to FIG. 3, medications M1 of the explained category C_{EX} and medications M2 of the unexplained category C_{UE} are located at different blocks.

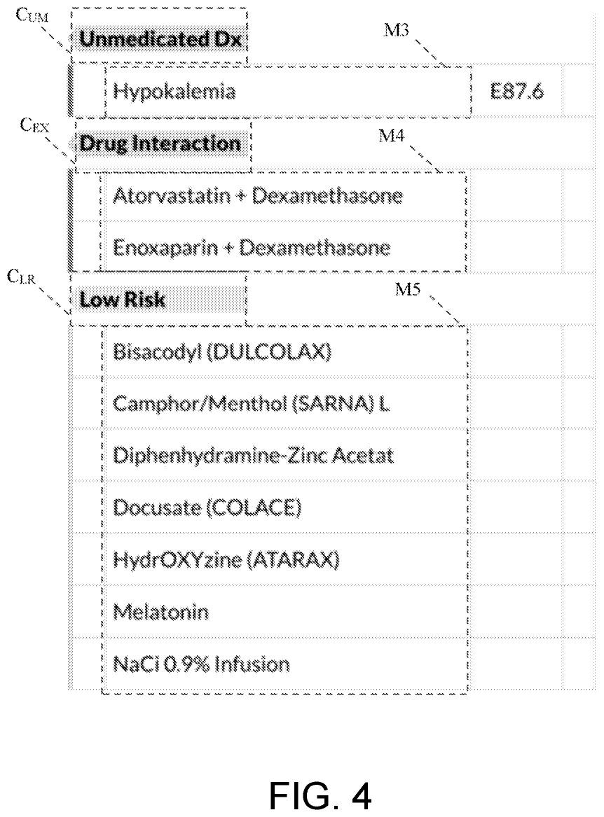

For another example, FIG. 4 is a schematic diagram illustrating medication categories according to one of the exemplary embodiments of the disclosure. Referring to FIG. 4, a medication M3 of the unmedicated category C_{UM}, medications M4 of the drug interaction category C_{DI}, and medications M5 of the low-risk category CLR are located at different blocks.

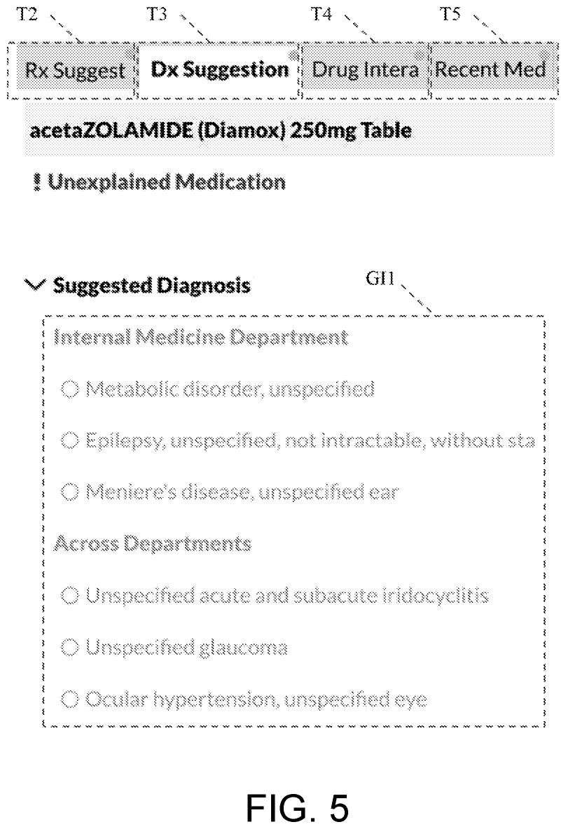

In one embodiment, one or more blocks are separated by one or more tabs within a single window. Taking FIG. 3 as an example, the explained category C_{EX} and the unexplained category C_{UE} are located in a tab T1 related to the association together. For another example, FIG. 5 is a schematic diagram illustrating guiding information related to unexplained medication according to one of the exemplary embodiments of the disclosure. Referring to FIG. 5, there are four tabs T2, T3, T4, and T5 for different categories.

In one embodiment, the processor 130 may configure multiple visual indications for multiple medication categories, respectively. The visual indications may relate to color, symbol, or pattern. For example, the medication of the explained category is shown with a gray background, and the medication of the unexplained category is shown with a yellow background. For another example, the medication of the unmedicated category is shown with a star symbol, and the drug interaction category is shown with an exclamation mark.

In one embodiment, the processor 130 may provide first guiding information on the medication list interface in response to a selection of the unexplained category. The first guiding information may relate to a suggested diagnosis. Taking FIG. 5 as an example, the processor 130 receives a selection operation by a user through an input device such as a touch panel, mouse, or keyboard. The selection operation relates to the selection of tab T3 for the unexplained category on the medication list interface. The guiding information GI1 related to the suggested diagnosis is presented on the medication list interface. Accordingly, a clinical person may be instructed with a proper diagnosis.

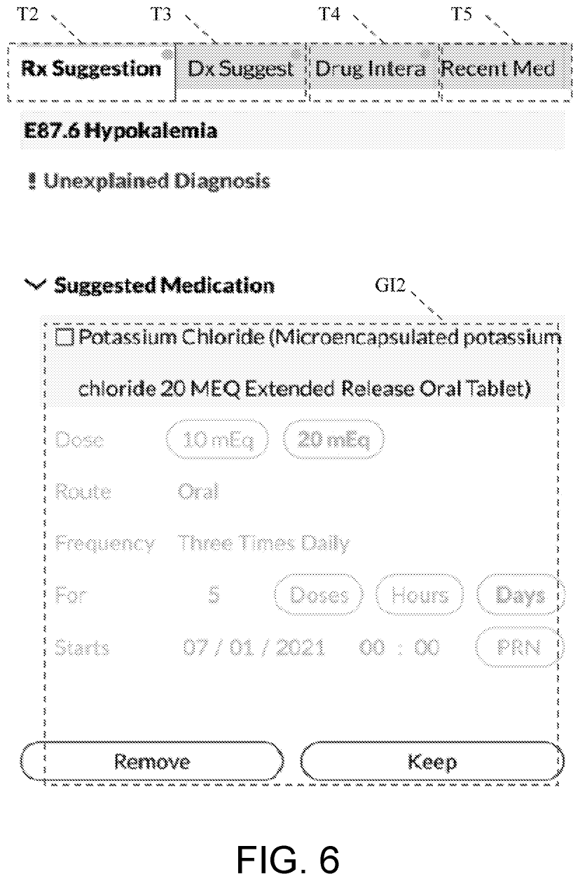

In another embodiment, the first guiding information relates to a deletion of the second medication belonging to the unexplained category in the medical record or an alternative option of the second medication. For example, FIG. 6 is a schematic diagram illustrating guiding information related to unexplained diagnosis according to one of the exemplary embodiments of the disclosure. Referring to FIG. 6, the selection operation of a user relates to the selection of tab T2 for the unexplained category on the medication list interface. The guiding information GI2 related to removing or keeping a specific medication with a specific dosage is presented on the medication list interface. Accordingly, a clinical person may be instructed with proper medication.

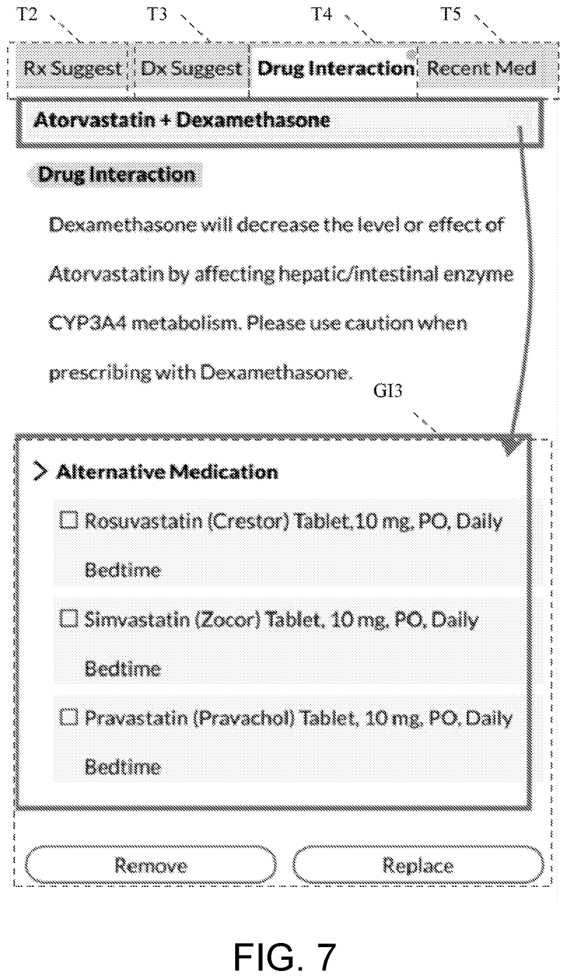

In one embodiment, the processor 130 may provide second guiding information on the medication list interface in response to a selection of the drug interaction category. The second guiding information relates to a dosage modification or an alternative option of a third medication belonging to the drug interaction category. For example, FIG. 7 is a schematic diagram illustrating guiding information related to drug interaction according to one of the exemplary embodiments of the disclosure. Referring to FIG. 7, the selection operation of a user relates to the selection of tab T4 for the drug interaction category on the medication list interface. The guiding information GI3 related to alternative medications is presented on the medication list interface. For another example, an item for a dosage addition or a dosage reduction of the third medication belonging to the drug interaction category may be shown on the medication list interface. Accordingly, the clinical risk may be reduced.

In some embodiments, other guiding information may be provided in response to a selection of another medication category.

In addition, the disclosure further provides a non-transitory computer-readable recording medium (e.g., a storage medium such as a hard disk, a compact disk, a flash memory, or a solid state disk (SSD)). The computer-readable recording medium is capable of storing a plurality of code segments (e.g., code segments of storage space detection, code segments of spatial adjustment option presentation, code segments of maintenance work, and code segments of frame presentation, etc.). After the code segments are loaded onto the processor 130 or another processor and executed, all the steps of the above method related to medication list management can be completed.

In summary, in the method, the apparatus related to medication list management, and the non-transitory computer-readable recording medium of the embodiment of the disclosure, more medication categories are integrated into the medical record. Accordingly, the medication history would be structured, grouped, and visual-encoded, so as to provide an intuitive medication list.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

1. A method related to medication list management, comprising:
determining medication association between at least one diagnosis and at least one corresponding medication recorded in a medical record through an evaluating model, wherein the evaluating model is trained through a machine learning algorithm;
integrating a plurality of medication categories into the medical record based on the medication association, wherein the at least one medication comprises at least one of a first medication and a second medication, and the plurality of medication categories comprise an explained category and an unexplained category, the explained category comprises the first medication having higher medication association with a corresponding diagnosis, and the unexplained category comprises the second medication having lower medication association with a corresponding diagnosis; and
providing a medication list interface presenting the medical record with the plurality of medication categories.

2. The method according to claim 1, wherein providing the medication list interface comprises:
providing first guiding information on the medication list interface in response to a selection of the unexplained category, wherein the first guiding information relates to a deletion of the second medication in the medical record or an alternative option of the second medication.

3. The method according to claim 1, wherein the plurality of medication categories further comprise an unmedicated category, and integrating the plurality of medication categories into the medical record based on the medication association comprises:
determining diagnosis that has no medication recorded in the medical record belongs to the unmedicated category.

4. The method according to claim 1, wherein the plurality of medication categories further comprise a drug interaction category, the at least one corresponding medication comprises a plurality of medications, and integrating the plurality of medication categories into the medical record based on the medication association comprises:
determining an interaction between the plurality of medications recorded in the medical record, wherein medication with the interaction relating to duplicating medication or lowering efficacy belongs to the drug interaction category.

5. The method according to claim 4, wherein providing the medication list interface comprises:
providing second guiding information on the medication list interface in response to a selection of the drug interaction category, wherein the second guiding information relates to a dosage modification or an alternative option of a third medication belonging to the drug interaction category.

6. The method according to claim 1, wherein the plurality of medication categories further comprise a low-risk category, and integrating the plurality of medication categories into the medical record based on the medication association comprises:
determining a side effect of the at least one medication recorded in the medical record, wherein medication having side effect with less harm belongs to the low-risk category.

7. The method according to claim 1, wherein providing the medication list interface comprises:
providing a plurality of blocks on the medication list interface, wherein each of the plurality of blocks corresponds to one of the plurality of medication categories.

8. The method according to claim 7, wherein the plurality of blocks are separated by a plurality of tabs within a single window.

9. The method according to claim 1, wherein providing the medication list interface comprises:
configuring a plurality of visual indications for the plurality of medication categories, respectively, wherein the plurality of visual indications relate to color, symbol, or pattern.

10. The method according to claim 1, wherein the unexplained category is divided into an unexplained medication sub-category and an unexplained diagnosis sub-category.

11. An apparatus related to medication list management, comprising:
a memory, storing a program code;
a display; and
a processor, coupled to the memory and the display, configured to load and execute the program code stored in the memory to perform:
determining medication association between at least one diagnosis and at least one corresponding medication recorded in a medical record through an evaluating model, wherein the evaluating model is trained through a machine learning algorithm;
integrating a plurality of medication categories into the medical record based on the medication association, wherein the at least one medication comprises at least one of a first medication and a second medication, and the plurality of medication categories comprise an explained category and an unexplained category, the explained category comprises the first medication having higher medication association with a corresponding diagnosis, and the unexplained category comprises the second medication having lower medication association with a corresponding diagnosis; and
providing a medication list interface presenting the medical record with the plurality of medication categories through the display.

12. The apparatus according to claim 11, wherein the processor is further configured for:
providing first guiding information on the medication list interface in response to a selection of the unexplained category, wherein the first guiding information relates to a deletion of the second medication in the medical record or an alternative option of the second medication.

13. The apparatus according to claim 11, wherein the plurality of medication categories further comprise an unmedicated category, and the processor is further configured for:
determining diagnosis that has no medication recorded in the medical record belongs to the unmedicated category.

14. The apparatus according to claim 11, wherein the plurality of medication categories further comprise a drug interaction category, the at least one corresponding medication comprises a plurality of medications, and the processor is further configured for:
determining an interaction between the plurality of medications recorded in the medical record, wherein medication with the interaction relating to duplicating medication or lowering efficacy belongs to the drug interaction category.

15. The apparatus according to claim 14, wherein the processor is further configured for:
providing second guiding information on the medication list interface in response to a selection of the drug interaction category, wherein the second guiding information relates to a dosage modification or an alternative option of a third medication belonging to the drug interaction category.

16. The apparatus according to claim 11, wherein the plurality of medication categories further comprise a low-risk category, and the processor is further configured for:
determining a side effect of the at least one medication recorded in the medical record, wherein medication having side effect with less harm belongs to the low-risk category.

17. The apparatus according to claim 11, wherein the processor is further configured for:
providing a plurality of blocks on the medication list interface, wherein each of the plurality of blocks corresponds to one of the plurality of medication categories.

18. The apparatus according to claim 17, wherein the plurality of blocks are separated by a plurality of tabs within a single window.

19. The apparatus according to claim 11, wherein the processor is further configured for:
configuring a plurality of visual indications for the plurality of medication categories, respectively, wherein the plurality of visual indications relate to color, symbol, or pattern.

20. A non-transitory computer-readable recording medium, recording a program code, the program code being loaded onto a processor to perform:
determining medication association between at least one diagnosis and at least one corresponding medication recorded in a medical record through an evaluating model, wherein the evaluating model is trained through a machine learning algorithm;
integrating a plurality of medication categories into the medical record based on the medication association, wherein the at least one medication comprises at least one of a first medication and a second medication, and the plurality of medication categories comprise an explained category and an unexplained category, the explained category comprises the first medication having higher medication association with a corresponding diagnosis, and the unexplained category comprises the second medication having lower medication association with a corresponding diagnosis; and
providing a medication list interface presenting the medical record with the plurality of medication categories.
